(19) United States
(12) Patent Application Publication
(10) Pub. No.: US 2014/0148447 A1
(43) Pub. Date: May 29, 2014

(54) AGENT FOR PROPHYLACTIC AND THERAPEUTIC TREATMENT OF HERPES INFECTIONS

(71) Applicant: Jan Slomianny, Numbrecht (DE)

(72) Inventor: Jan Slomianny, Numbrecht (DE)

(21) Appl. No.: 14/167,676

(22) Filed: Jan. 29, 2014

Related U.S. Application Data
(63) Continuation of application No. 13/639,357, filed on Feb. 19, 2013.

(30) Foreign Application Priority Data
Apr. 8, 2010 (DE) 1020100142905

(51) Int. Cl. A61K 31/5415

(52) U.S. Cl. CPC A61K 31/5415
USPC 514/226.5

(57) ABSTRACT

The invention relates to an agent for the prophylactic and therapeutic treatment of virus infections, said agent containing piroxicam in a carrier substance.

The invention relates to an agent for the prophylactic and therapeutic treatment of virus infections, e.g. herpes infections, in particular involving herpes simplex.

The treatment of virus infections in humans and animals has always been a great challenge due to the fact that only a limited number of active agents is available and this is also true for the family of herpesviruses.

The family of Herpesviridae comprises a great number of viruses having a double-stranded DNA. Widely spread are herpes simplex viruses of type HSV 1 and HSV 2 as well as the herpes varicella zoster virus VZV. All cause painful infections in the form of superficial inflammation. Herpesviruses remain dormant within the human body for a long time so that the outbreaks of the disease will occur repeatedly and may even display serious symptoms.

Herpes simplex infections will primarily affect the mouth and manifest themselves in the form of blisters and lesions on mucous membranes and lips. Varicella zoster infections manifest themselves during childhood mostly in the form of chickenpocks, and in grownup humans in the form of shingles. The latter will produce painful exanthema in the region of a spinal nerve, for example in the region of the loins, the thorax but even in the face. Zoster infections are accompanied by fever, loss of appetite, aching limbs and aches in the area of the exanthema.

Various agents were developed against herpesviruses but these are mainly effective in that they alleviate the symptoms and generally have merely a limited influence on the course of the disease. In most cases, their use only results in marginally shortening the duration of the disease. An active agent frequently employed in this context is aciclovir.

Basically, there is a need for agents suited to effectively counteract virus infections, in particular also herpes infections.

Publication EP 1 457 202 A2 describes the use of so-called non-steroidal anti-inflammatory drugs (NSAIDs) for the treatment of herpes infections. Although the application mentioned a great variety of NSAIDs their effectiveness has only been described for two members of this group, i.e. diclofenac and ketorolac, and substantial proof based on reliable data could be furnished only for diclofenac. According to these data, topically administered diclofenac was suited to alleviate the course of the disease, healing of lesions appeared to take five days on average. This meant although the normal infection duration of up to 10 days could thus be shortened, the situation was nevertheless dissatisfactory for the patients.

Surprisingly, it has now been found that an active agent, piroxicam, which belongs to the group of NSAIDs is suited for the prophylactic and therapeutic treatment of virus infections. Piroxicam has a positive influence on a variety of viral diseases, for example influenza cases, caused by influenza viruses of type H₁N₁, and is in particular suited to prevent or bring about a quick healing process of herpes infections, i.e. those of types HSV1 and HSV2, especially herpes labialis. Piroxicam, 4-hydroxy-2-methyl-N-pyridine-2-yl-2H-1,2-benzothiazine-3-carboxamide-1,1-dioxide, is a COX inhibitor and used as antirheumatic drug.

Accordingly, the invention relates to an agent of the kind first mentioned above that comprises piroxicam in a suitable carrier substance.

According to the invention the agent preferably contains piroxicam in an amount of 0.1 to 10% w/w, preferably 0.1 to 5 and especially preferred in an amount ranging between 0.5 and 5% w/w. It may be administered topically, orally or parenterally.

The inventive agent is preferably employed for the treatment of infections in the region of the mouth, commonly known by the term herpes labialis. These are infections resulting from the herpes simplex virus HSV 1 or HSV 2, with the agent also being effective against herpes zoster VZV. All the aforesaid infections cause superficial painful exanthemas.

As a rule, the agent is topically administered, particularly in the form of cream, ointment or tincture. These contain customary carrier substances, that is formulations for creams, ointments, gels and tinctures established for use in medical practice.

Moreover, the agent may also be administered in the form of tablets, powder, solutions to be infused or injected.

The agent is used by patients 1 to 5 times, 1 to 2 times daily. The majority of the patients said that a single use of the agent had been successful.

Many patients frequently suffering from herpes simplex infections reported that applying the agent prophylactically in stress situations which typically give rise to the formation of herpes exanthemas prevented the occurrence of such exanthemas.

### TEST REPORT 1

Making use of a commercially available piroxicam gel with an active agent content of 0.5% w/w the inventive agent was tested on 42 subjects with results achieved as follows:

Of the 42 test persons 26 used the agent once and 16 subjects used it up to five times.

15 of the 42 subjects noted signs of amelioration in less than one day (which also included that outbreaks of the disease could be prevented), 21 subjects reported amelioration in one to three days and two an amelioration in four to ten days. Tolerability of the agent was reported by 41 of the test persons as good and one subject stated tolerability to be not so good. One subject reported the agent had not helped.

The effectiveness of piroxicam actually came as a surprise in this context. The active agent, originally developed as antirheumatic drug and noted chemically correct as 4-hydroxy-2-methyl-N(2-pyridinyl)-2H-1,2-benzothiazine-3-carboxamide-1,1-dioxide, is still administered as an analgesic. In chemical respect, it is completely different from the NSAIDs diclofenac and ketorolac described hereinbefore so that—due to its chemical structure—a virucidal effect on herpesviruses and in particular with regard to herpes simplex/herpes labialis cannot be expected. In contrast to diclofenac and keterolac the agent is suited to prevent the occurrence of exanthemas.

### TEST REPORT 2

In a screening test a piroxicam-containing gel with an active agent content of 0.4% was tested on cultures infected by herpesviruses of type HSV1 adopting standard testing methods. Initially, the CD50-value was found to be 2.50, with the virus titer being 7.00 in the beginning. After a residence time of 1, 5 and 60 min. the virus titer (log₁₀ TCID₅₀/ml) was found to be lower than 2.50 corresponding to a reduction of the virus count by more than 99.99%.

1. A method of prophylactic and therapeutic treatment of a virus infection caused by a virus of the family of herpesviridae, the method comprising
administering to a patient in need of such treatment an agent in an amount effective to treat the virus infection, wherein the agent contains piroxicam in a carrier substance.

2. The method according to claim 1, wherein the agent is characterized by a content of piroxicam ranging between 0.1 and 10% w/w.

3. The method according to claim 2, wherein the agent is characterized by a content of piroxicam ranging between 1 and 5% w/w.

4. The method according to claim 1 characterized in that the virus infection is an infection caused by herpes simplex.

5. The method according to claim 1, wherein the administering is by topical administration.

6. The method according to claim 4, wherein the agent is in the form of a cream, ointment, tincture or gel.

7. The method according to claim 1, wherein the administering is by infusion or injection.

8. The method according to claim 4, wherein the virus infection is an infection caused by herpes labialis.
